(19) United States
(12) Patent Application Publication
(10) Pub. No.: US 2023/0160886 A1
(43) Pub. Date: May 25, 2023

(54) QUALITY ANALYSIS NANOSENSOR USING METASTRUCTURE

(71) Applicant: KOREA FOOD RESEARCH INSTITUTE, Wanju-gun, Jeollabuk-do (KR)

(72) Inventors: Gyoengsik OK, Hwaseong-si, Gyeonggi-do (KR); Dongman KIM, Yongin-si, Gyeonggi-do (KR); Youngjin CHO, Jeonju-si, Jeollabuk-do (KR); Seokin HONG, Seoul (KR)

(21) Appl. No.: 17/972,957

(22) Filed: Oct. 25, 2022

Related U.S. Application Data
(63) Continuation of application No. PCT/KR2021/005292, filed on Apr. 27, 2021.

(30) Foreign Application Priority Data
Apr. 27, 2020 (KR) 10-2020-0050916

(51) Int. Cl. G01N 33/543; G01N 33/02; G01N 27/327; G01N 27/414

(52) U.S. Cl. CPC G01N 33/5438; G01N 33/02; G01N 27/3278; G01N 27/4145

(57) ABSTRACT

Proposed is a quality analysis nanosensor using a metastructure, including: a metasurface structure resonating with a specific frequency of incident electromagnetic waves; a fixed binding body formed on a surface of the metasurface structure or inside the metasurface structure on a hotspot area; a movable binding body coupled to the fixed binding body by an attractive force; and a receptor or nanoparticles linked to the movable binding body. According to the nanosensor, there are provided a detection structure and method based on metamaterials and nanoparticles, thereby enabling efficient detection with only few nanoparticles by raising detection sensitivity to a high level.

### TECHNICAL FIELD

The disclosure relates to a quality analysis nanosensor using a metastructure, and more particularly to a quality analysis nanosensor based on a metamaterial, in which detection sensitivity is efficiently raised to a high level with only few nanoparticles.

### BACKGROUND ART

Biosensing technology refers to analysis technology based on a biosensor. To systematically explain the biosensing technology, it is necessary to look at what the biosensor includes. The biosensor largely includes a transducer and a biological element, in which the transducer detects variations in ions, electrons, heat, mass, and light, resulting from a selective reaction between the biological element and an analyte, coverts the variations into electric signals, and amplifies the electric signals into reaction signals. Therefore, depending on the characteristics of the transducer, the biosensor is broadly classified into an ‘electrochemical biosensor’ for detecting variations in electrical properties, an ‘optoelectronic biosensor’ for detecting variations in optical properties, an ‘piezoelectric biosensor’ for detecting variations in mass, and a ‘biothermistor’ for detecting thermal variations in resulting from a bioreaction.

The biosensors have been mainly applied to the fields of medicine, food & agriculture, processes, environments, and the like. A biosensor market size is rapidly growing in the field of food, and the use of the biosensor in the food industry is also expected to increase in the future. In terms of technology, the electrochemical biosensor has the highest share.

In the food industry, the biosensing technology is applicable to the fields such as ingredient analysis, rapid detection of natural toxins and antinutrients, detection of enzyme inactivation and microbial contamination during food processing and food preservation, measurement of hazardous substances generated during a cooking process or by interaction between food ingredients, production of food raw ingredient, analysis of contaminants mixed during processing, measurement of fish freshness, evaluation of antioxidant activity or the like functionality, and fermentation monitoring.

In addition, a biosensor for assessing the freshness by measuring relative proportions of major substances produced while fish meat and livestock meat are decomposing, a biosensor for evaluating antioxidant activity or the like functionality, a biosensor for accessing a food process and measuring the concentration of fermentation products online in real time, etc., are highly applicable in the food industry.

As the biosensor market size is rapidly growing in the field of food, it can be said that the future of food biosensing technology is very bright. Further, the development of proteomics and the like omics technology is promoting the research, development and application of food biosensors.

In the future, there will be a surge in demand for a disposable biosensor or a simple, cost-effective, quick-response and easy-to-use biosensor device. Accordingly, the standardization and miniaturization of biosensor chips are essential to improve reproducibility and reduce costs. Ultimately, it is necessary to develop the biosensing technology for food based on micro-total analysis systems (μTAS) and establish peripheral element technology for this.

In the case of a conventional metamaterial using a nanogap, a nanogap-based metamaterial sensor could be used as a more sensitive sensor due to a field enhancement (FE) effect at the nanogap.

However, it is difficult for the current level of technology to actually apply the nanogap to a low-cost sensor because a manufacturing process is complicated and costs high.

When nanoparticles are bound onto the metamaterial, detection sensitivity is significantly amplified, but there are difficulties such as inefficiency in the case of a big unit cell of metamaterials, and necessity of many nanoparticles.

Further, when simple label-free measurement is performed without a biochemical selective binding site even though the metamaterial is used, detection is possible but inefficient.

### DISCLOSURE

### Technical Problem

The disclosure is conceived to solve the foregoing problems, and an aspect of the disclosure is to provide a detection structure and method based on metamaterials and nanoparticles, which enable efficient detection with only few nanoparticles while raising detection sensitivity to a high level.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1A is a schematic diagram showing a structure of a quality analysis nanosensor using a metastructure according to the disclosure.

FIGS. 1B to 1D show various pattern shapes formed in metasurface structures and examples of hotspot areas.

FIGS. 1E and 1F are schematic diagrams showing the structure and detection mechanism of the nanosensor according to embodiments of the disclosure.

FIG. 2A is a schematic diagram showing a simulation of label-free sensing, in which the detection is carried out as a detection target material is uniformly adsorbed to the surface of the metastructure shown in FIG. 1D and increased in mass per unit area.

FIG. 2B is a graph showing the results of finite difference time domain analysis for a metastructure sensor, the entire metastructure surface of which is coated with Al₂O₃ particles of FIG. 2A, and FIG. 2C is a graph showing the results of peak shift effects versus variations in the number of particles.

FIG. 3A is a schematic diagram showing that Al₂O₃ particles are adsorbed only to a certain local area of a metastructure unit cell shown in FIG. 1D, and FIGS. 3B and 3C are graphs showing transmittance according to movement of centric coordinates.

FIGS. 4 and 5 are graphs showing changes in image and transmittance peaks according to movement of island positions of particles, respectively.

FIG. 6A is a graph showing a peak shift versus variations in the number of particles according to an embodiment where Al₂O₃ particles are formed in a hotspot area of the metastructure corresponding to (a) of FIG. 4, and FIG. 6B is a graph showing the results of peak shift effects according to this embodiment.

FIG. 7A is a graph showing the results of finite difference time domain analysis according to an embodiment where polyelectrolyte complex (PEC) particles combined with second magnetic particles are formed in the hotspot area of the metastructure shown in FIG. 1D, and FIG. 7B is a graph showing the results of peak shift effects according to this embodiment.

### BEST MODE

According to an aspect of the disclosure, there is provided a quality analysis nanosensor using a metastructure, including: a metasurface structure resonating with a specific frequency of incident electromagnetic waves; a fixed binding body formed on a surface of the metasurface structure or inside the metasurface structure on a hotspot area; a movable binding body coupled to the fixed binding body by an attractive force; and a receptor or nanoparticles linked to the movable binding body.

Further, the hotspot area may include an area where a field enhancement phenomenon for strongly concentrating intensity of an electric field occurs.

Further, the fixed binding body may include first magnetic particles including one selected from the group consisting of ferromagnetic metals such as nickel, iron, cobalt, and rare earth compounds, or a mixture thereof, and the movable binding body may include second magnetic particles employing one selected from the group consisting of ferromagnetic metals such as nickel, iron, cobalt, and rare earth compounds, or a mixture thereof; or magnetoplasmonic particles obtained by combining one selected from the group consisting of ferromagnetic metals or a mixture thereof with silver or gold nanoparticles, and bound to the first magnetic particles by an attractive force.

Further, the fixed binding body may include a chemical linker including single, double or multiple ionic ligands with derivatives of sulfur (S), nitrogen (N), and oxygen (O) and the movable binding body may include particles employing metal or nonmetal nanoparticles combined with one or more selected from the group consisting of carbohydrate, peptide, protein, enzyme, lipid, amino acid, deoxyribonucleic acid (DNA), ribonucleic acid (RNA), antibody, polyethylene glycol (PEG), drug, and fluorescent dye, and bound to the chemical linker.

Further, the chemical linker is formed on the surface of the structure or inside the structure on the hotspot area by lithography.

Further, the receptor may be formed with a binding site to which a target material for detecting the quality of an analyte is specifically bound.

### [Mode for Invention]

The disclosure relates to a quality analysis nanosensor using a metastructure, which includes a metasurface structure resonating with a specific frequency of incident electromagnetic waves; a fixed binding body formed on a surface of the metasurface structure or inside the metasurface structure on a hotspot area; a movable binding body coupled to the fixed binding body by an attractive force; and a receptor or nanoparticles linked to the movable binding body.

Below, the disclosure will be described in detail with reference to the accompanying drawings.

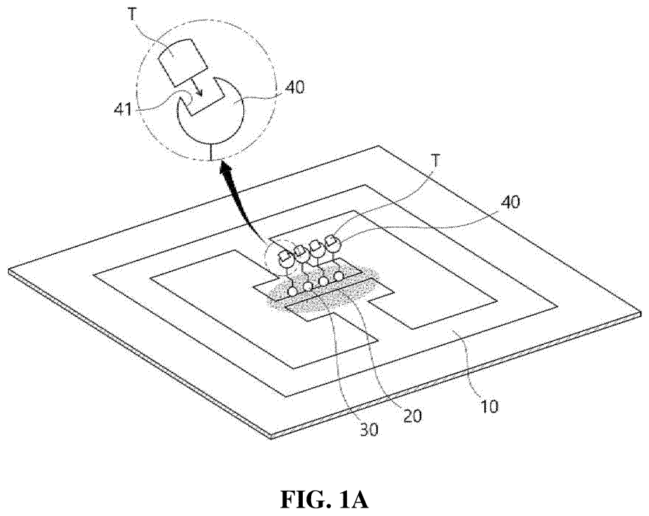

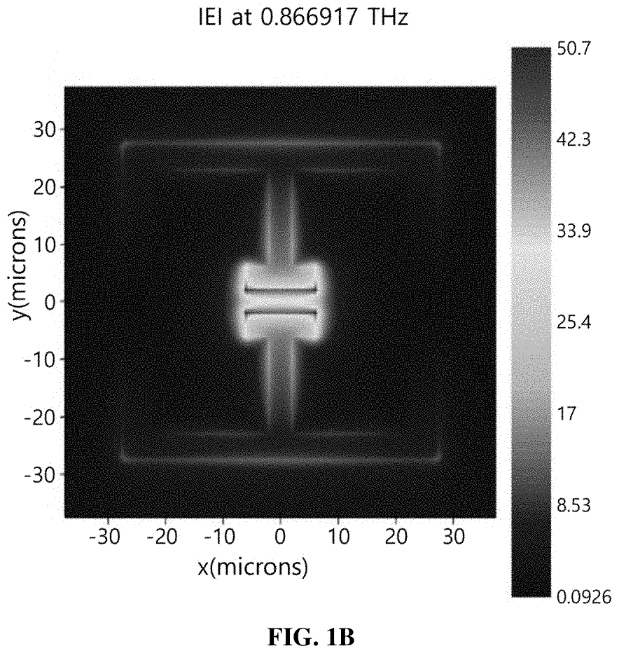

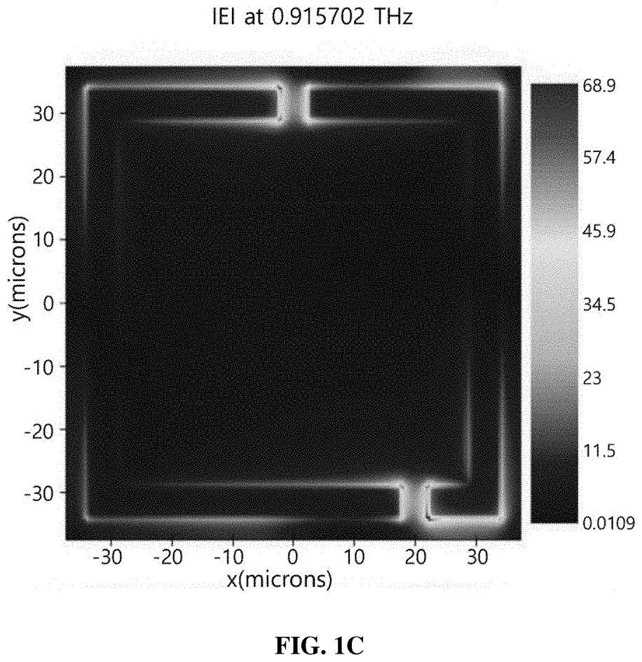

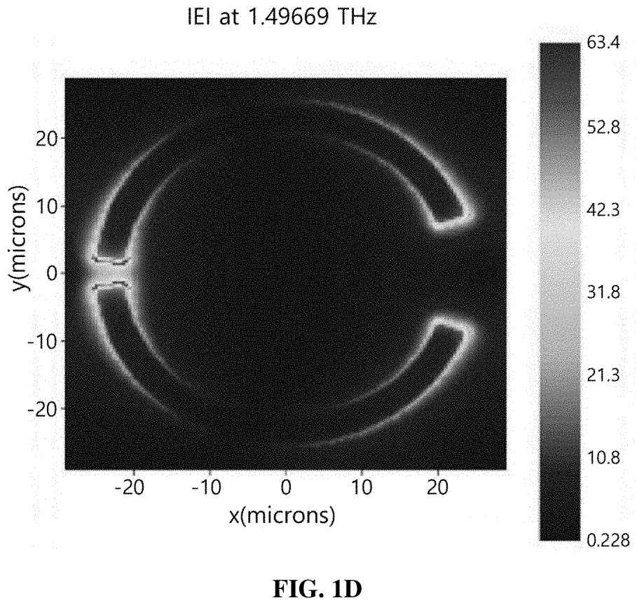

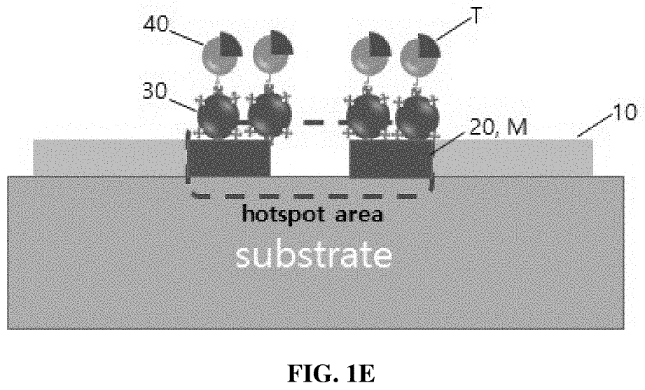

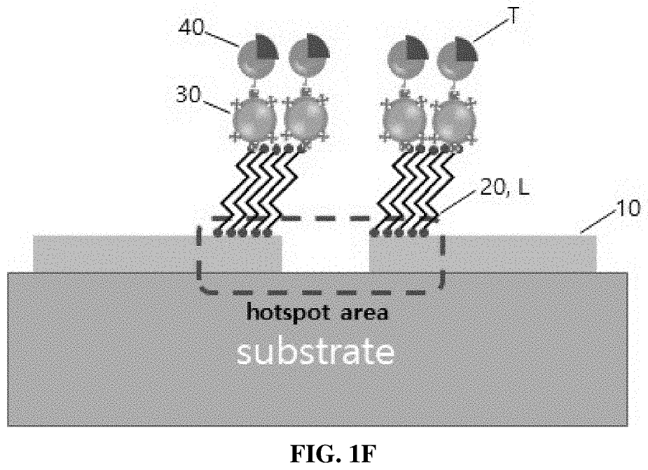

FIG. 1A is a schematic diagram showing a structure of a quality analysis nanosensor using a metastructure according to the disclosure, the structure includes:

a metasurface structure 10 that resonates with a specific frequency of incident electromagnetic waves;

a fixed binding body 20 formed on a surface of the metasurface structure 10 or inside the structure on a hotspot area;

a movable binding body 30 coupled to the fixed binding body 20 by an attractive force; and

a receptor 40 or nanoparticles linked to the movable binding body 30.

In a metamaterial unit cell, a position of a hotspot, where a field effect (FE) occurs, is varied depending on the structures. FIGS. 1B to 1D show pattern shapes formed in various metasurface structures and examples of hotspot areas according to the pattern shapes.

For example, in the case of a resonance structure of a representative split ring resonator such as an electric-field coupled inductor-capacitor (ELC) resonator shown in FIG. 1B, the hotspot area is formed in a middle capacitor portion. In the case of asymmetric resonance structures shown in FIGS. 1C and 1D, the hotspot area is formed at edge portions.

In the metastructure sensor according to an embodiment of the disclosure, a plane, on which meta patterns are formed, i.e., the metasurface structure 10 is used as a base, and first magnetic particles 20 are formed on the pattern plane or at specific position inside the pattern, thereby improving detection sensitivity.

FIG. 1E shows an example that the fixed binding body 20, i.e., the first magnetic particles M are introduced in an area, in which the hotspot is generated, within the metamaterial pattern according to an embodiment of the disclosure. Referring to FIGS. 1A and 1E, the first magnetic particles M, which includes ferromagnetic metals (Ni, Fe, etc.) or an alloy thereof, may be introduced into the hotspot area among the metamaterial patterns.

Then, the movable binding body 30, i.e., second magnetic particles, which includes a magnetic metal or the like, may be introduced onto the metamaterial surface in the form of flowing as contained in a fluid. The second magnetic particles 30, i.e., the magnetic metal may be used in the form of nanoparticles. In this way, when the magnetic nanoparticles are mixed into the fluid and flow on the surface of the metasurface structure 10, the magnetic nanoparticles are highly likely to be collected near the hotspot selectively formed in the surface of the metasurface structure 10.

The second magnetic particles 30 are linked to the receptor 40 or the nanoparticles.

In this case, the receptor 40 or the nanoparticles are formed with a binding site 41 to be specifically bound to a target material T, and thus a specific target material T for detecting the material quality is bound to the binding site 41. Therefore, all the nanomagnetic particles in the fluid are concentrated and attached to the magnetic pattern of the hotspot area with little loss, and the number of binding sites per unit area of the fixed binding body increases, thereby enhancing the sensitivity. Accordingly, the target material T attached to the binding site 41 of the receptor 40 or nanoparticles is positioned within the hotspot area, thereby greatly improving an efficiency of detecting the quality of the analyte.

In this case, when the second magnetic particles 30 have a dual function of magnetoplasmonic particles combined with nanoparticles of gold, silver or the like, stronger adsorption occurs, thereby enabling high-sensitivity measurement.

FIG. 1F shows an example that the fixed binding body 20, i.e., a chemical linker L is introduced into a hotspot area of a metamaterial pattern according to another embodiment of the disclosure. The chemical linker L includes single, double or multiple ionic ligands with derivatives of sulfur (S), nitrogen (N), and oxygen (O). As particles to be bound to the chemical linker L, the movable binding body 30 may be introduced with a fluid, which may employ metal or nonmetal nanoparticles combined with one or more selected from the group consisting of carbohydrate, peptide, protein, enzyme, lipid, amino acid, deoxyribonucleic acid (DNA), ribonucleic acid (RNA), antibody, polyethylene glycol (PEG), drug, and fluorescent dye.

The detection mechanism of the foregoing embodiment is similar to that of an embodiment employing the first magnetic particles and the second magnetic particles, in which the target material T specifically bound to the binding site of the receptor or nanoparticles linked to the movable binding body by combination between the chemical linker and the movable binding body is concentrated in a specific hotspot area, thereby improving the detection sensitivity.

Below, embodiments of the disclosure will be described in detail.

### Embodiments

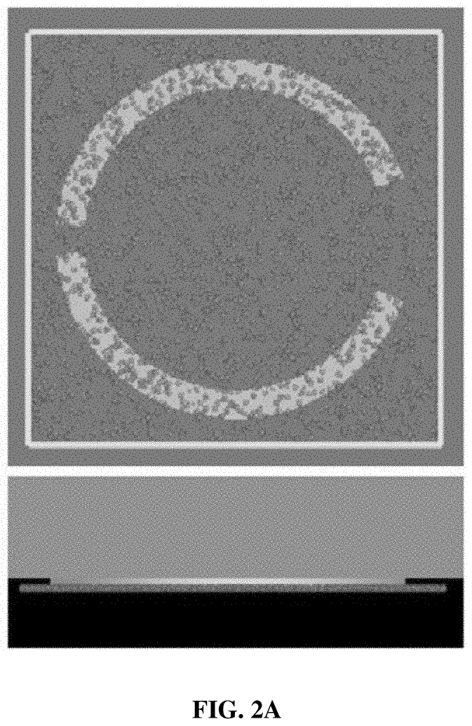

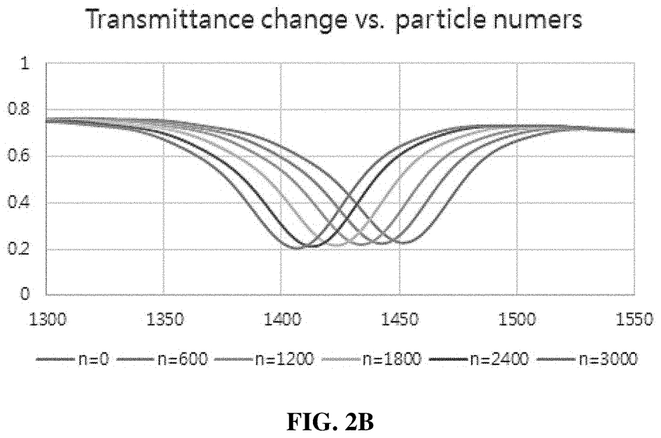

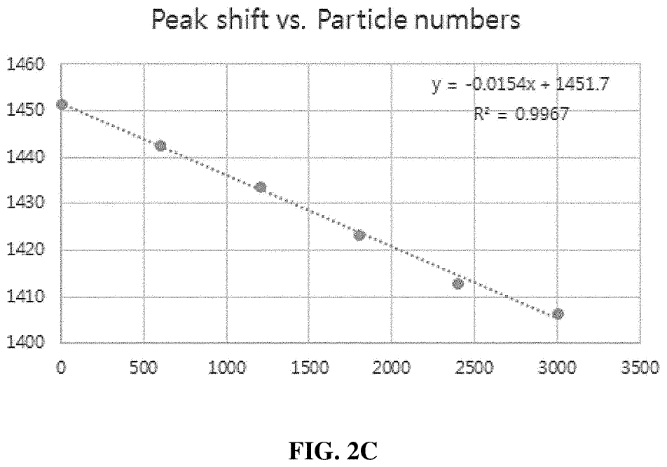

FIG. 2A is a schematic diagram showing a simulation of label-free sensing, in which the detection is carried out as a detection target material is uniformly adsorbed to the surface of the metastructure shown in FIG. 1D and increased in mass per unit area. In particular, the schematic diagram shows the metastructure, the entire surface of which is uniformly coated with Al₂O₃ particles (diameter of 0.8 to 1.0 μm, n=3.07), which are used as an example of dielectric materials having a higher refractive index than general biomaterials, so as to maximize the effect of label-free sensing (top: the front of a unit cell, and bottom: the side of a unit cell, where a metal layer (i.e., a yellow green pattern) was emphasized thicker than the actual one, and the unit cell has a size of 58 μm×58 μm, and a metal pattern has a line width of 4 μm). FIG. 2B is a graph showing the results of finite difference time domain analysis for a metastructure sensor, the entire metastructure surface of which is coated with the Al₂O₃ particles of FIG. 2A, the graph showing the transmittance varied as the number of nanoparticles increases. FIG. 2C is a graph showing change in peak shift effects versus variations in the number of particles in the transmittance graph of FIG. 2B. A calculation result shows that a resonant frequency (peak) is red-shifted by change in mass as the number of particles per unit cell surface of the metastructure increases, as well known in general label-free sensing. In this case, the peak shift versus the variations in the number of particles shows a change of 15.4 GHz per 1000 particles based on a linear change.

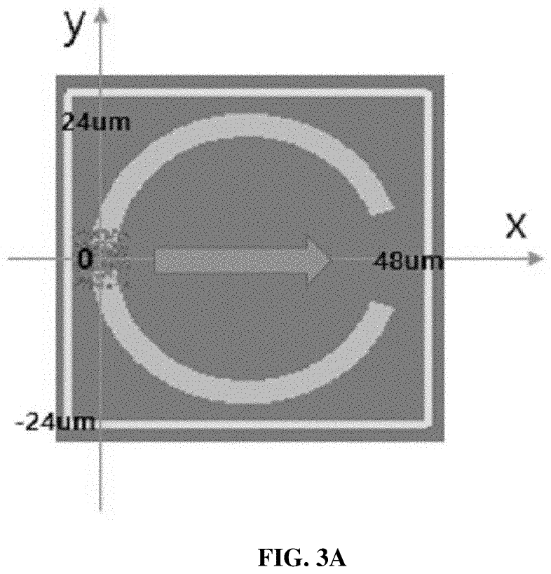

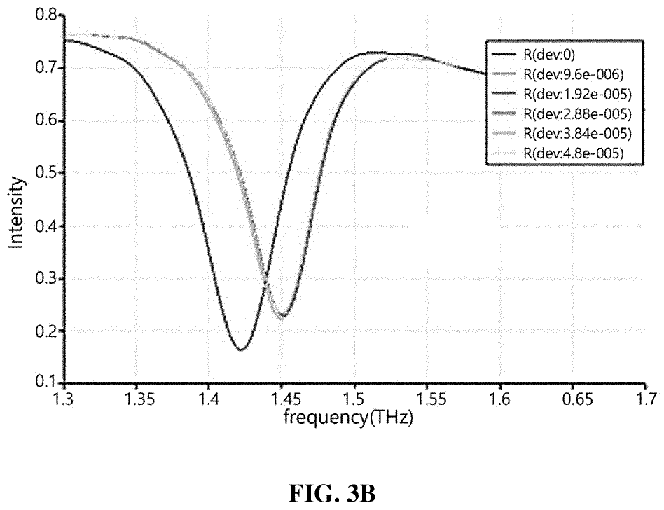

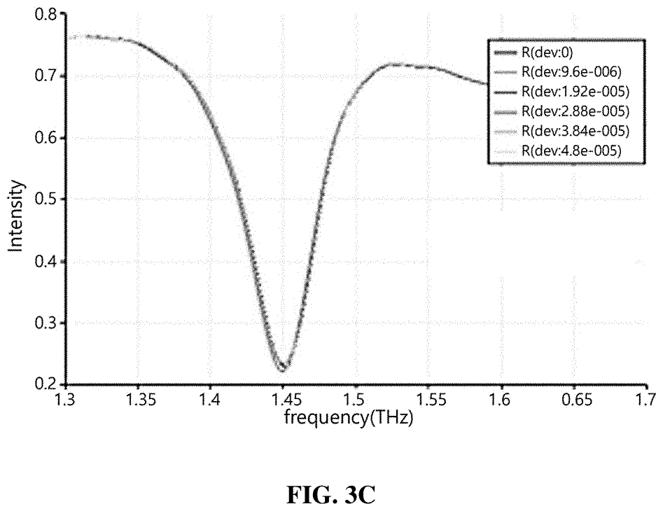

FIG. 3A is a schematic diagram for calculation of when Al₂O₃ particles are adsorbed only to a certain local area (i.e., an island area: 10 μm×10 μm) of a metastructure unit cell shown in FIG. 1D, and FIG. 3B shows the transmittance of when the y-centric coordinate of the island is 0 and the x-centric coordinate is moved from 0 to 48 μm. As expected, the results of FIG. 3B show that more adsorption occurs when the particles are concentrated nearer the hotspot, and spectra are almost similar outside the hotspot area. Further, FIG. 3C shows the transmittance of when the y-centric coordinate of the island is 24 μm and the x-centric coordinate is moved from 0 to 48 μm. The results of FIG. 3C show the transmittance of the island of the particles adsorbed to an area other than the hotspot, and the results show that there is little change in spectra throughout the areas even though the X-centric coordinates of the island are changed. In the case of FIG. 3B, there is large change in peak between when the particles are near the hotspot and when the particles are not near the hotspot. On the contrary to the case of FIG. 3B, in the case of FIG. 3C, there is little change in peak because not all areas are the hotspot. These results show that the mass change of the particle island can be expressed with higher sensitivity when the particles are adsorbed nearer the hotspot.

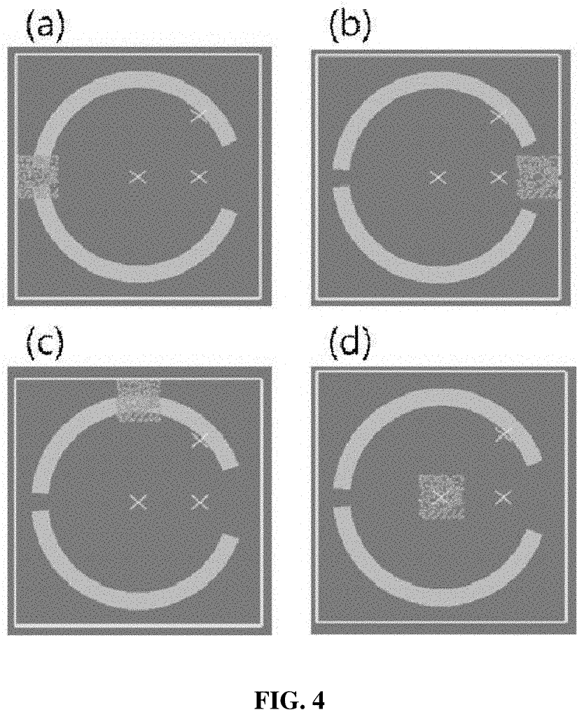

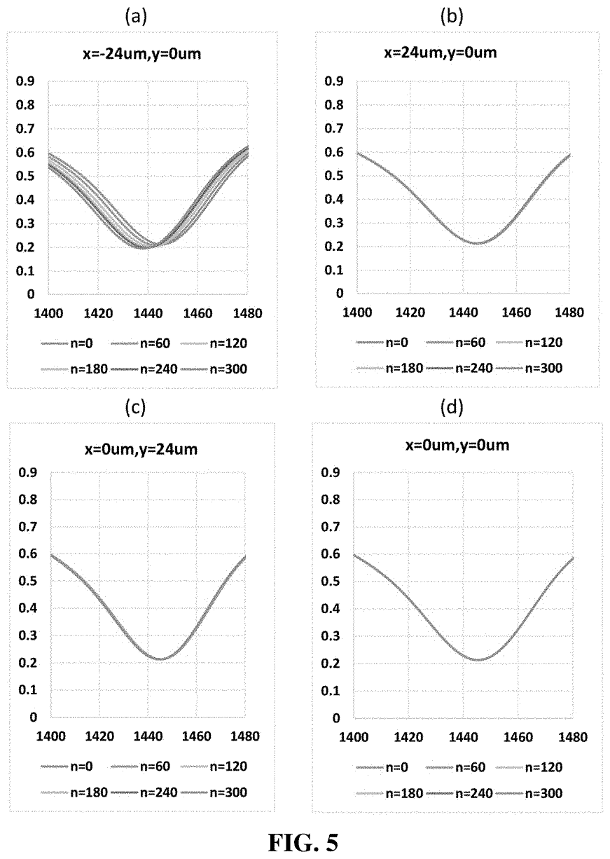

To examine these results in more detail, FIGS. 4 and 5 ((a) to (d)) show change in the transmittance peak versus variations in the number of adsorbed particles (e.g., 0 to 300 particles) while moving the position of the particle island from the hotspot area (a: x=−24 μm, y=0 μm) to the non-hotspot area (b: x=24 μm, y=0 μm, c: x=0 μm, y=24 μm, d: x=0 μm, y=0 μm). As expected, the results show that the peak shift versus the variations in the number of particles is observed only in the hotspot area (see (a) of FIG. 5), but there is little movement or no change in the other areas.

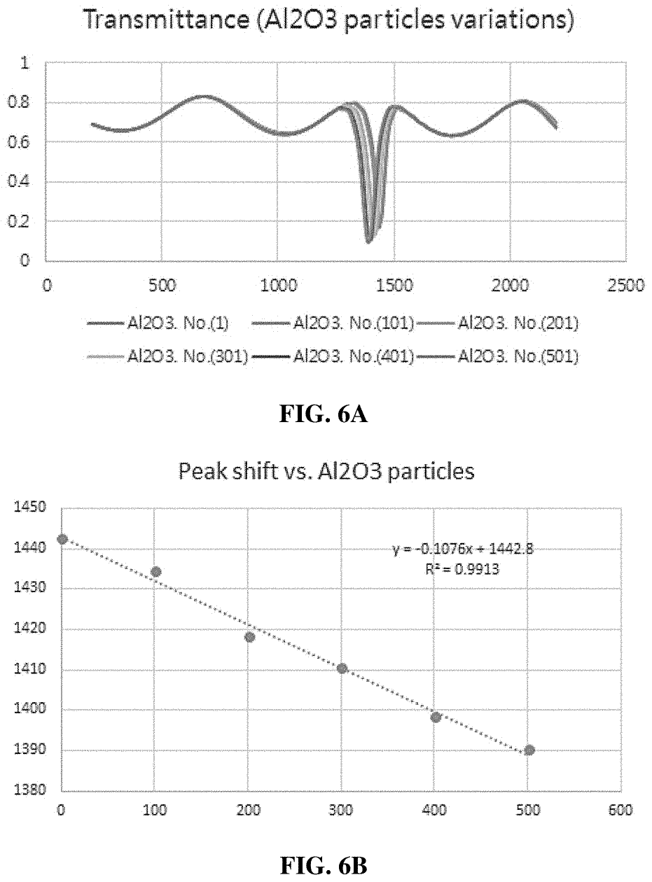

FIG. 6A is a graph showing a peak shift versus variations in the number of particles according to an embodiment where Al₂O₃ particles are formed in the hotspot area (y=0) of the metastructure corresponding to (a) of FIG. 4, i.e., a graph showing the results of finite difference time domain analysis for quantifying the sensitivity (Al₂O₃ particles (n=3.07), variations (1 to 501 particles)). FIG. 6B is a graph showing the results of peak shift fitting. A calculation result shows a change of 107 GHz/1000 particles as shown in FIG. 6B, which is more amplified 7 times than the change of FIG. 2C. Taking such results together, much higher sensitivity is obtained when particles are concentrated and adsorbed within the hotspot area. Based on this principle, it will be appreciated that, when the first magnetic particles 20 are introduced into the area where the hotspot is generated, the sensitivity is significantly increased as the second magnetic particles are adsorbed near the hotspot.

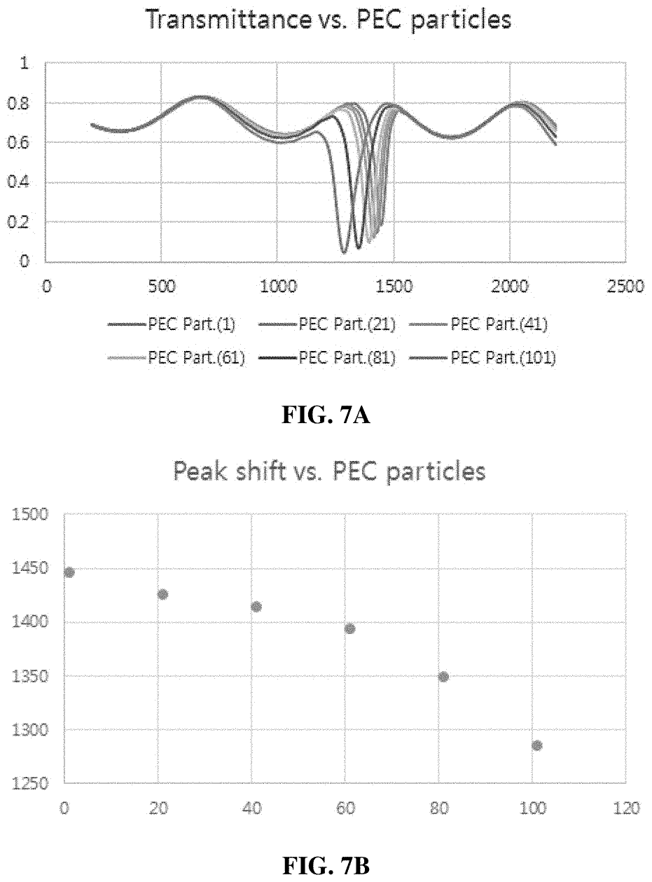

FIG. 7A is a graph showing the results of finite difference time domain analysis according to an embodiment where polyelectrolyte complex (PEC, using metal nanoparticles instead of the foregoing dielectric materials) particles combined with second magnetic particles are formed in the hotspot area of the metastructure shown in FIG. 1D (PEC particles, variations (1 to 101 particles)). FIG. 7B shows peak shift effects. The result shows a sensitivity of 160 GHz/100 particles, which is more amplified about 15 times than that of the foregoing case, and shows that the sensitivity is rapidly increased as the magnetoplasmonic particles obtained by combining gold nanoparticles and magnetic particles are adsorbed.

From such results, the magnetic pattern is formed in the hotspot area regardless of the area of the metamaterial unit cell so that the magnetic particles for the detection can be concentrated in a specific area, thereby enabling highly sensitive measurement with only the biosensor attached to few magnetic particles.

Further, stronger adsorption occurs when the magnetoplasmonic particles are used as the second magnetic particles, thereby further amplifying the sensitivity.

### REFERENCE NUMERALS

10: metasurface structure

20: fixed binding body

30: movable binding body

40: receptor

41: binding site

T: target material

M: first magnetic particles

L: chemical linker

### INDUSTRIAL APPLICABILITY

According to the disclosure, there is provided a nanosensor for detecting the quality, which has a detection structure based on metamaterials and nanoparticles, thereby enabling efficient detection with only few nanoparticles by raising detection sensitivity to a high level.

1. A quality analysis nanosensor using a metastructure, comprising:
a metasurface structure resonating with a specific frequency of incident electromagnetic waves;
a fixed binding body formed on a surface of the metasurface structure or inside the metasurface structure on a hotspot area;
a movable binding body coupled to the fixed binding body by an attractive force; and
a receptor or nanoparticles linked to the movable binding body.

2. The quality analysis nanosensor of claim 1, wherein the hotspot area comprises an area where a field enhancement phenomenon for strongly concentrating intensity of an electric field occurs.

3. The quality analysis nanosensor of claim 1, wherein
the fixed binding body comprises first magnetic particles comprising one selected from the group consisting of ferromagnetic metals such as nickel, iron, cobalt, and rare earth compounds, or a mixture thereof, and
the movable binding body comprises second magnetic particles employing one selected from the group consisting of ferromagnetic metals such as nickel, iron, cobalt, and rare earth compounds, or a mixture thereof; or magnetoplasmonic particles obtained by combining one selected from the group consisting of ferromagnetic metals or a mixture thereof with silver or gold nanoparticles, and bound to the first magnetic particles by an attractive force.

4. The quality analysis nanosensor of claim 1, wherein
the fixed binding body comprises a chemical linker comprising single, double or multiple ionic ligands with derivatives of sulfur (S), nitrogen (N), and oxygen (O), and
the movable binding body comprises particles employing metal or nonmetal nanoparticles combined with one or more selected from the group consisting of carbohydrate, peptide, protein, enzyme, lipid, amino acid, deoxyribonucleic acid (DNA), ribonucleic acid (RNA), antibody, polyethylene glycol (PEG), drug, and fluorescent dye, and bound to the chemical linker.

5. The quality analysis nanosensor of claim 4, wherein the chemical linker is formed on the surface of the structure or inside the structure on the hotspot area by lithography.

6. The quality analysis nanosensor of claim 1, wherein the receptor is formed with a binding site to which a target material for detecting the quality of an analyte is specifically bound.
